(19) United States
(12) Patent Application Publication
(10) Pub. No.: US 2009/0191132 A1
(43) Pub. Date: Jul. 30, 2009

(54) Coded medication and methods of preparing same for identification and distinguishment

(76) Inventor: Joseph A. Sniadach, Baltimore, MD (US)

Correspondence Address: J. Bruce Hoofnagle, P.O. Box 370, Lisbon, MD, 21765-0370, US

(21) Appl. No.: 12/322,021

(22) Filed: Jan. 28, 2009

Related U.S. Application Data
(60) Provisional application No. 61/062,650, filed on Jan. 28, 2008.

(51) Int. Cl. A61K 31/495; A61J 1/14

(52) U.S. Cl. 424/10.3; 604/404

(57) ABSTRACT

A selected substance is dissolved into a given injectable fluid medication having an initial clear visual appearance. The selected substance is selected from a group of substances which, when dissolved in the injectable fluid medication, will alter the clear appearance to produce a non-clear or altered visual appearance. The altered visual appearance could be a color, which, by code, identifies the given injectable fluid medication, and distinguishes the given injectable fluid medication from a different injectable fluid medication, the clear visual appearance of which has been altered by dissolving another selected substance to produce a colored visual appearance different from the color of the altered visual appearance of the given injectable fluid medication.

This application claims the benefit of U.S. Provisional Application No. 61/062,650, filed Jan. 28, 2008, which is incorporated herein by reference thereto.

### BACKGROUND OF THE INVENTION

This invention relates to coded medications and methods of preparing same for identification and distinguishment. More particularly, this invention relates to identifiable coded injectable fluid medications, and to methods of preparing same, and to different coded injectable fluid medications to distinguish such medications, one from the other, and to methods of preparing same, for use in the treatment of patients, and to reduce and eliminate medication-selection errors, which have occurred in the past, and which could be potentially traumatic to patients, to whom the erroneously selected medications are to be administered.

Medication errors remain a significant threat to human health. There are currently many different organizations that monitor medications and associated errors resulting in significant patient morbidity and mortality. The Food and Drug Administration of the U.S. Department of Health and Human Services (“FDA”) has campaigned to reduce and eliminate medication errors. This reduction in medication errors has been addressed in the handling and administration of medications, which included inappropriate labeling and abbreviations. Drug mix-ups of similar appearing medications have resulted in death. For example, as noted in The FDA Patient Safety News, June 2007, a clear liquid diuretic and a clear liquid cardiovascular medication had similar vial labels and caps, which lead to an incorrect selection of medication, resulting in cardiac arrhythmia in the patient following the administering of the incorrectly selected medication. The majority of injectable medications are clear, and are not distinguishable after the medication is drawn into a syringe or a bag of clear intravenous fluid.

The Joint Commission International Center for Patient Safety has extensively addressed medication errors. Strategies to reduce medication errors are multi-factorial, which includes handling, human error, mislabeling and misreading of handwriting and abbreviations. According to a report released by the Institute of Medicine, “between 44,000 and 98,000 deaths may result may result each year from medical errors in hospitals alone. And more than 7,000 deaths each year are related to medications.” Again the magnitude of this issue has resulted in recent FDA proposals for bar codes on drugs and user-friendly format labeling for physicians to reduce errors and subsequent adverse effects, including death.

Oral medications have some identification, such as, for example, color, shape, wording, and numbering. However, injectable medications, with few exceptions, are all clear when provided in a vial, or other form of container, which may have an identification label. However, once a clear injectable medication is removed from its identifying vial or container, the medication looks no different than other clear medications, whether in an unlabelled vial, a syringe or an intravenous fluid bag.

The only way, heretofore, to identify the clear medication is to label, in some way, its container such as, for example a vial, a syringe and an intravenous fluid bag, which, as we know, has lead to medication error, be it from mental distraction by the medical provider, mislabeling, etc. The main areas of focus for unmistakable identification of clear injectable medications include operating rooms, emergency rooms, coronary care units, and intensive care units, where potent respiratory and cardiac medications are used. In addition, such errors can occur in facilities, such as hospital and public pharmacies, from which pharmaceuticals are ultimately supplied at the direction of a physician.

Injectable medication mistakes from improper labeling have resulted in cardiac and respiratory arrest in all of the above-cited areas due to overdosing of narcotics, electrolytes, such as potassium, and benzodiazepine overdose to cite a few examples. Myocardial infarction, hypertensive crisis and fatal arrhythmias have also occurred from mistaken administration of cardiac stimulants and vasopressors such as epinephrine, phenylephrine and ephedrine.

In addition, the operating room, with related anesthesia events, provides an additional and unique arena for clear injectable medication error, where busy and overworked anesthetists and anesthesiologists inject potent respiratory and cardiovascular medications many times daily. Many times, the hurried provider withdraws a clear medication from a labeled vial, but does not label a syringe, or draws a second medication into an empty syringe which has already been labelled with another medication. In addition to the aforementioned adverse affects, a unique situation of having a patient awake and aware during surgery can occur where a muscle relaxant is inappropriately dosed.

Thus, there is a constant need for improvement in eliminating errors in the selection, and administering, of injectable medications for the safety and life of the recipients of the medications.

### SUMMARY OF THE INVENTION

It is, therefore, an object of this invention to provide a coded injectable fluid medication which is identifiable, and methods of identifying the coded injectable fluid medication, by the visual appearance thereof.

Another object of this invention is to provide coded injectable fluid medications for, and methods of, distinguishing between at least two different injectable fluid medications by the visual appearances of the medications.

With these and other objects in mind, this invention contemplates a coded injectable fluid medication identifiable by a coded visual appearance thereof, for use in the treatment of patients. An injectable fluid medication has a prescribed efficacy and an initial visual appearance, and is selected from the groups of pharmaceuticals consisting of barbiturates, benzodiazepines, opioids, ketamine, etomidate and derivatives thereof, local anesthetics, muscle relaxants, cardiovascular medications, antihistamines, anticholinergics, anticholinesterases, antidotes, antitoxins, electrolytes, anticoagucants and reversal agents, thrombolytics, butyrophenones, antibiotics, antiseizure medications, chemotherapeutics, contrast media, and radiologic agents. A selected substance is dissolved in the injectable fluid medication, without altering the prescribed efficacy of the injectable fluid medication, to produce an altered visual appearance of the injectable fluid medication which is different from the initial visual appearance. The selected substance is selected from any one or more substances of a group of substances which will alter the visual appearance of the injectable fluid medication, and which has been approved by the Food and Drug Administration of the U.S. Department of Health and Human Services for injection into human beings.

This invention further contemplates a first coded injectable fluid medication and a second coded injectable fluid medication, which is a different medication from the first coded injectable fluid medication, coded by the visual appearances thereof, for distinguishing the first coded injectable fluid medication from the second coded injectable fluid medication, for use in the treatment of patients. The first injectable fluid medication has a prescribed efficacy and an initial visual appearance, and is selected from the groups of pharmaceuticals consisting of barbiturates, benzodiazepines, opioids, ketamine, etomidate and derivatives thereof, local anesthetics, muscle relaxants, cardiovascular medications, antihistamines, anticholinergics, anticholinesterases, antidotes, antitoxins, electrolytes, anticoagucants and reversal agents, thrombolytics, butyrophenones, antibiotics, antiseizure medications, chemotherapeutics, contrast media, and radiologic agents. A selected substance is dissolved in the first injectable fluid medication, without altering the prescribed efficacy of the injectable fluid medication, to produce an altered visual appearance of the first injectable fluid medication which is different from the initial visual appearance of the first injectable fluid medication. The selected substance is selected from any one or more substances of a group of substances which will alter the visual appearance of the injectable fluid medication, and which have been approved by the Food and Drug Administration of the U.S. Department of Health and Human Services for injection into human beings The second injectable fluid medication has a prescribed efficacy and an initial visual appearance which is different from the altered visual appearance of the first injectable fluid medication, where the second injectable fluid medication is selected from the groups of pharmaceuticals consisting of barbiturates, benzodiazepines, opioids, ketamine, etomidate and derivatives thereof, local anesthetics, muscle relaxants, cardiovascular medications, antihistamines, anticholinergics, anticholinesterases, antidotes, antitoxins, electrolytes, anticoagucants and reversal agents, thrombolytics, butyrophenones, antibiotics, antiseizure medications, chemotherapeutics, contrast media, and radiologic agents.

This invention also contemplates the provision of a first coded injectable fluid medication and a second coded injectable fluid medication, which is a different medication from the first coded injectable fluid medication, coded by the visual appearances thereof, for distinguishing the first coded injectable fluid medication from the second coded injectable fluid medication. The selected substance, noted in the preceding paragraph, is a first selected substance, and a second selected substance, different from the first selected substance, is dissolved in the above-noted second injectable fluid medication. The dissolving of the second selected substance does not alter the prescribed efficacy of the second injectable fluid medication, and produces an altered visual appearance of the second injectable fluid medication which is different from the initial visual appearance of the second injectable fluid medication, and different from the altered visual appearance of the first injectable fluid medication. The second selected substance is selected from any one or more substances of a group of substances which will alter the visual appearance of the second injectable fluid medication, and which have been approved by the Federal Drug Administration of the U.S. Department of Health and Human Services for injection into human beings.

In addition, this invention contemplates the provision of a method of preparing a coded injectable fluid medication identifiable by the visual appearance thereof. This method includes providing an injectable fluid medication having a prescribed efficacy and an initial visual appearance, which is selected from the groups of pharmaceuticals consisting of barbiturates, benzodiazepines, opioids, ketamine, etomidate and derivatives thereof, local anesthetics, muscle relaxants, cardiovascular medications, antihistamines, anticholinergics, anticholinesterases, antidotes, antitoxins, electrolytes, anticoagucants and reversal agents, thrombolytics, butyrophenones, antibiotics, antiseizure medications, chemotherapeutics, contrast media, and radiologic agents. The method further includes providing a selected substance which, when dissolved in the injectable fluid medication, without altering the prescribed efficacy of the injectable fluid medication, will produce an altered visual appearance of the injectable fluid medication which is different from the initial visual appearance, where the selected substance is selected from any one or more substances of a group of substances which will alter the visual appearance of the injectable fluid medication, and which have been approved by the Federal Drug Administration of the U.S. Department of Health and Human Services for injection into human beings. The method also includes dissolving the selected substance in the injectable fluid medication to produce the altered visual appearance of the injectable fluid medication.

This invention further contemplates the provision of a method for preparing a first coded injectable fluid medication and a second coded injectable fluid medication, which is a different medication from the first coded injectable fluid medication, and for distinguishing the first coded injectable fluid medication from the second coded injectable fluid medication, by the visual appearances thereof, for use in the treatment of patients. This method includes providing the first injectable fluid medication, which has a prescribed efficacy and an initial visual appearance, and is selected from the groups of pharmaceuticals consisting of barbiturates, benzodiazepines, opioids, ketamine, etomidate and derivatives thereof, local anesthetics, muscle relaxants, cardiovascular medications, antihistamines, anticholinergics, anticholinesterases, antidotes, antitoxins, electrolytes, anticoagucants and reversal agents, thrombolytics, butyrophenones, antibiotics, antiseizure medications, chemotherapeutics, contrast media, and radiologic agents. Further, this method includes providing a selected substance which, when dissolved in the first injectable fluid medication, without altering the prescribed efficacy of the injectable fluid medication, will produce an altered visual appearance of the first injectable fluid medication which is different from the initial visual appearance of the first injectable fluid medication. The selected substance is selected from any one or more substances of a group of substances which will alter the visual appearance of the injectable fluid medication, and which have been approved by the Federal Drug Administration of the U.S. Department of Health and Human Services for injection into human beings. This method also includes providing the second injectable fluid medication, which has a prescribed efficacy, and an initial visual appearance, which is different from the altered visual appearance of the first injectable fluid medication. The second injectable fluid medication is selected from the groups of pharmaceuticals consisting of barbiturates, benzodiazepines, opioids, ketamine, etomidate and derivatives thereof, local anesthetics, muscle relaxants, cardiovascular medications, antihistamines, anticholinergics, anticholinesterases, antidotes, antitoxins, electrolytes, anticoagucants and reversal agents, thrombolytics, butyrophenones, antibiotics, antiseizure medications, chemotherapeutics, contrast media, and radiologic agents.

This invention also contemplates the provision of a method, wherein the selected substance, noted in the preceding paragraph, is a first selected substance. This method includes providing a second selected substance, different from the first selected substance. The second selected substance is dissolvable in the second injectable fluid medication, without altering the prescribed efficacy of the second injectable fluid medication, and will produce an altered visual appearance of the second injectable fluid medication which is different from the initial visual appearance of the second injectable fluid medication, and different from the altered visual appearance of the first injectable fluid medication. The second selected substance is selected from any one or more substances of a group of substances which will alter the visual appearance of the second injectable fluid medication, and which have been approved by the Federal Drug Administration of the U.S. Department of Health and Human Services for injection into human beings. The method further includes dissolving the second selected substance in the second injectable fluid medication to produce the altered visual appearance of the second injectable fluid medication, which is different from the altered visual appearance of the first injectable fluid medication.

Other objects, features and advantages of the present invention will become more fully apparent from the following detailed description of the preferred embodiment and the appended claims.

### BRIEF DESCRIPTION OF THE DRAWINGS

In the accompanying drawings:

FIG. 1 is a perspective view showing a prior art two-compartment intravenous bag;

FIG. 2 is a sectional view showing a prior art two-compartment vial; and

FIG. 3 is a partial sectional view showing the prior art vial of FIG. 2.

### DESCRIPTION OF THE PREFERRED EMBODIMENT OF THE INVENTION

For many years, in the current system of providing the many different injectable medications used for medical treatment of patients, pharmaceutical companies have manufactured the different injectable medications with a clear visual appearance. The clear medications are placed in a container such as, for example, a vial, an ampule, a pre-filled syringe, an intravenous bag, or the like prior to shipping the medications to pharmacies and hospitals. However, without some form of identification, each clear medication, in each container, would be indistinguishable from the many remaining different containerized medications with a clear visual appearance.

In an effort to identify and distinguish the different clear injectable medications, the pharmaceutical companies have used various techniques externally of the container to identify and distinguish the many clear medications. Such techniques include placing a pre-printed label on each container, with the name of the medication printed on the label, printing bar codes on the label unique to the medication within the container, and using labels, and/or container caps, of different colors based on a color code scheme unique to the medication within the containers.

In preparation for, and during, a medical procedure for a patient, an anesthesiologist, or any medical professional, i.e., a physician, a nurse, or a paramedic, will draw up different clear injectable medications into respectively different syringes, or into a common syringe, labeled or unlabeled. In similar fashion, intravenous bags may be prepared prior to, or during, the medical procedure. Once the clear medications have been withdrawn from the labeled containers, the ability to identify and distinguish between different syringe-loaded, or intravenous bag-loaded, medications is lost.

In an attempt to avoid mistakes in the use of such medication-loaded syringes and bags, the anesthesiologists, or medical professionals, typically place a section of tape on the syringes and bags, and may hand-write, on the tape, the name of the drug. Even then, mistakes occur resulting in potential harm to the patient.

The above-described, currently available techniques and facilities, which are external to the medication, i.e., on the outside of the container, syringe and/or intravenous bag, are prone to human error, and require time-consuming reading and analysis by the handlers and users thereof, which, in many crucial situations, is time that they can ill afford.

Therefore, after many years of relying on the error-prone external techniques and facilities, as described above, to identify and distinguish between injectable medications in containers provided by the pharmaceutical manufacturers, and/or in syringes and intravenous bags prepared on site, the invention described and claimed herein presents a new and unique approach, wherein the different injectable medications are identified and distinguishable by the color of the medications. The handlers and professionals who come in contact with these colored injectable medications will be able to immediately identify and distinguish the different injectable medications by the visual appearance of the medications, internally of the container, rather than having to rely on the time-consuming reading and/or analyzation of the above-noted external facilities currently in use.

The visually clear, injectable medications, which are referred to hereinafter as “injectable fluid medications,” under this invention, are included in several groups of pharmaceuticals, and all such injectable fluid medications have a prescribed efficacy. The pharmaceuticals in these several groups include barbiturates, benzodiazepines, opioids, ketamine, etomidate and derivatives thereof, local anesthetics, muscle relaxants, cardiovascular medications, antihistamines, anticholinergics, anticholinesterases, antidotes, antitoxins, electrolytes, anticoagulants and its reversal agents, thrombolytics, butyrophenones, antibiotics, antiseizure medications, chemotherapeutics, contrast media, and radiologic agents.

Some examples of visually clear, injectable fluid medications in each of these groups are listed below. Any visually clear, injectable fluid medications not listed below as examples, which are known to be in any one of the above-listed groups, are covered by the claims of this application. Each of the following groups of pharmaceuticals is listed and enumerated, followed by the examples of the visually clear, injectable fluid medications in each group:
1. barbiturates—phenobarbital, pentobarbital;
2. benzodiazepines—midazolam, valium;
3. opioids—morphine, fentanyl;
4. ketamine;
5. etomidate and derivatives;
6. local anesthetics—lidocaine, bupivicaine, ropivicaine;
7. muscle relaxants—succinylcholine, rocuronium, vecuronium;
8. cardiovascular medications—epinephrine, ephedrine, lopressor;
9. antihistamines—benadryl, promethazine;
10. anticholinergics—atropine, scopolamine, glycopyrrolate;
11. anticholinesterases—neostigmine, pyridostigmine;
12. antidotes—narcan, flumazenil;
13. anticoagulants and its reversal agents—heparin, protamine;
14. electrolytes—calcium, potassium, magnesium;
15. thrombolytics—urokinase, reteplase;
16. butyrophenones—haloperidol, droperidol;
17. antibiotics—pencillin, gentamicin;
18. antiseizure—phenytoin;
19. chemotherapeutics—cyclophosphamide, carboplatin;
20. contrast media—diatrizoate meglumine, iodixanol;
21. radiologic agents—tositumomab, regadenoson.

In the manner described below, a single known substance, or a combination of such known substances, are dissolved in the clear injectable fluid medications to alter the visual appearance from an initial, or clear, appearance to an altered, non-clear, or colored appearance. Each substance, which is also referred to below as a composition, is selected from a group of known substances which, when dissolved in a clear medication, or in water, will alter the visual appearance of the medication by producing a colored, or a non-clear, appearance unique to the substance, or unique to a combination of such substances.

The substances, selected from the above-noted group, will not alter the above-noted prescribed efficacy of the injectable fluid medications, when dissolved in the medications. All of the substances, selected from the above-noted group of substances, have been approved by the FDA for injection into human beings. While the preferred form of the selected substances is powder, the form of the substances may also be liquid or solid without departing from the spirit and scope of the invention.

Representative substances, approved by the FDA, in the above-noted group of substances, from which a selection can be made for producing a particular non-clear, or color, visual appearance, when dissolved in the injectable fluid medications, include, but are not limited to, such as compositions as indigotindisulfonate sodium, which produces a deep blue color; rocuronium bromide, which produces a yellow/orange color; methylene blue, which produces a blue color; pemetrexed disodium, which produces light yellow or green-yellow color; sodium ferric gluconate complex, which produces a deep red color; hydroxocobalamin, which produces a dark red color; and indocyanine green, which produces a green color.

Any two or more of the color-producing substances approved by the FDA, including the above-noted substances, can be combined to produce other colors for use in altering the visual appearance of the clear injectable fluid medications.

The technique of coloring injectable fluid medications, as described and claimed herein, can be used in conjunction with a color code scheme wherein the visual appearance of each clear injectable fluid medication is altered with an assigned color unique to that medication, which is different from the colors assigned to the other medications, to identify individual medications, and to distinguish two or more different medications.

A coded injectable fluid medication, is identifiable by the visual appearance thereof, and includes as a portion thereof an injectable fluid medication, selected from the above-noted group of pharmaceuticals. The injectable fluid medication has a prescribed efficacy and an initial visual appearance, such as, for example, a clear appearance.

An appearance-altering substance, approved for injection into human beings by the FDA, is selected from any one or more of the above-noted group of substances, which, when dissolved in the injectable fluid medication, will produce an altered visual appearance, different from the initial visual appearance of the medication, and without altering the prescribed efficacy of the medication. The substance is selected on the basis that the substance will alter the initial visual appearance of the injectable fluid medication to an altered visual appearance, which is unique to the medication in accordance with a coded scheme.

In order to obtain the desired altered visual appearance, a prescribed amount of the selected substance is dissolved in a prescribed amount of the clear injectable fluid medication, with the prescribed amounts of the substance and medication being determined by a prescribed ratio of the amounts of the substance and the medication necessary to produce the desired non-clear, or colored, visual appearance.

The selected substance is then dissolved in the injectable fluid medication, whereby the initial visual appearance is altered to a non-clear appearance indicative of the injectable fluid medication in accordance with the coded scheme. As an example, when the selected substance is dissolved in the initial clear appearance of the injectable fluid medication, the initial clear visual appearance of the medication is altered to a non-clear, or colored, visual appearance, which is unique to the medication in accordance with the code scheme.

The above-noted initial visual appearance of the injectable fluid medication can be a clear appearance and the altered visual appearance thereof can be a non-clear appearance, which could be a colored appearance, all without departing from the spirit and scope of the invention. In addition, as noted above, the selected substance can be selected from a group of any one, or a mixture of two or more, compositions, which, when dissolved in the injectable fluid medication will change the initial visual appearance of the injectable fluid medication to a selected non-clear, or colored appearance. Further, as noted above, the form of the selected substance can be a fluid, a powder, or be solid.

Preferably, the selected substance is dissolved in the injectable fluid medication at the manufacturing site of the pharmaceutical company. In this manner, large volumes of the injectable fluid medication can be treated with the dissolved substance to provide the large volume of coded medication. Measured amounts of the coded medication can then be dispensed, in a conventional manner, into measured containers, such as vials, ampules, syringes, intravenous bags, and the like. The measured containers can then be shipped to receiving locations such as pharmacies, hospitals, and the like.

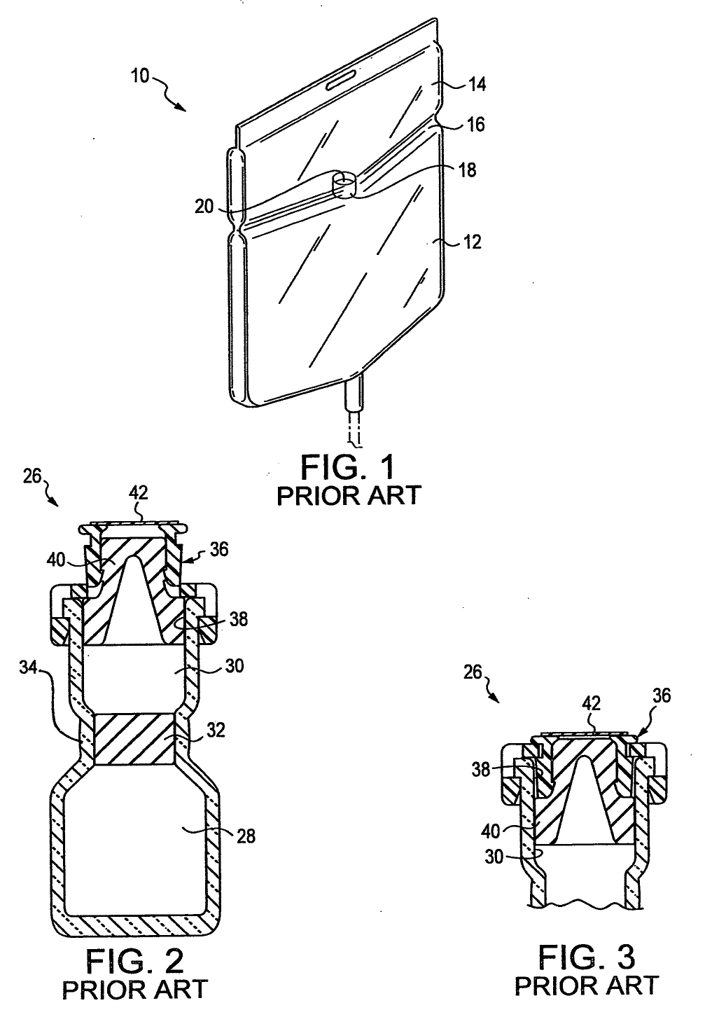

As illustrated in FIG. 1, a container, such as a prior art intravenous bag 10, is formed with a first compartment 12 and a second compartment 14, which is segregated from the first compartment by a divider wall 16. A flexible sleeve 18 is located within the divider wall 16 to facilitate communication between the first compartment 12 and the second compartment 14, but precludes such communication by a breakable seal 20, which is located within the sleeve. By squeezing the sleeve 18, externally of the bag 10, the seal 20 is broken to allow communication between the first compartment 12 and the second compartment 14. The prior art intravenous bag 10 is illustrated and described in U.S. Pat. No. 5,853,388, which is incorporated herein by reference thereto.

As illustrated in FIGS. 2 and 3, a container, such as a prior art vial 26, is formed with a first compartment 28 and a second compartment 30, which are sealed from communication therebetween by a compliant plug 32 located removably in a central neck 34 of the vial. A closure structure 36 is mounted on an open end 38 of the vial 26, and includes a resilient flexible stopper 40, which fits into, and seals, the open end of the vial. A dust seal 42 is located at an outer top of the closure structure 36, and is normally in an inactivated position as shown in FIG. 2. By pressing on the outer top surface of the dust seal 42, to move the dust seal toward the vial 26 to a position shown in FIG. 2, the stopper 40 is moved farther into the first compartment 28 of the vial to compress the volume of the first compartment. As the volume of the first compartment 28 is being compressed, the force of compression is applied against the stopper 40 to move the stopper into the second compartment 30, whereby the first compartment is in communication with the second compartment. The prior art vial 26 is illustrated and described in U.S. Pat. No. 4,258,845, which is incorporated herein by reference thereto.

As an alternative to fully preparing the coded injectable fluid medication at the pharmaceutical companies, a container having a first compartment and a second compartment, such as, for example, the prior art containers 10 and 26 described above, can be used. Such a container would be formed with a first compartment and a second compartment, which can be segregated by a releasable seal.

With the seal not in place, a prescribed amount of the selected substance is loaded into the first compartment, and the seal is put in place to segregate the first compartment from the second compartment. A prescribed amount of the injectable fluid medication is deposited into the second compartment. Means are attached to the container for facilitating opening of the seal, so that the selected substance dissolves in the injectable fluid medication to produce the altered visual appearance thereof. The opening, or breaking, of the seal can be effected at any desired location, such as, for example, the site of injecting the medication into the patient.

Whether the selected substance is dissolved in the injectable fluid medication at the pharmaceutical facilities, or at any other location, such as, for example, a pharmacy or hospital, a coded injectable fluid medication is formed, and is identifiable by the altered visual appearance.

The principle of the above-described coded injectable fluid medication can be used to distinguish between, or amongst, several injectable fluid medications. The description below will be directed to the distinguishment between a first coded injectable fluid medication and a second coded injectable fluid medication, which is a different medication from the first injectable fluid medication. It is understood that below-described principle regarding distinguishing between two medications, can be used to distinguish amongst three or more medications, without departing from the spirit and scope of the invention.

A selected substance is dissolved in a first injectable fluid medication in the manner described above, with respect to the identification of the medication, to produce an altered visual appearance. A second injectable fluid medication is selected from the above-noted groups of pharmaceuticals, and has a prescribed efficacy and an initial visual appearance, which is different from the altered visual appearance of the first injectable fluid medication. The first injectable fluid medication is distinguished from the second injection fluid medication by having an altered visual appearance which is different from the initial visual appearance of the second injectable fluid medicine.

With respect to the above-noted distinguishment between the first medication and the second medication, the initial visual appearances of the first and second injectable fluid medications are each a clear visual appearance.

Further, with respect to the above-noted distinguishment between the first medication and the second medication, the selected substance which was dissolved in the first injectable fluid medication is a first selected substance. A second selected substance, different from the first selected substance, is dissolved in the second injectable fluid medication, without altering the efficacy of the second fluid medication, to produce an altered visual appearance which is different from the initial visual appearance of the second injectable medication, and different from the altered visual appearance of the first injectable fluid medication, to provide distinguishing altered visual appearances between the first and second injectable fluid medications. The second selected substance is selected from any one or more substances of the above-noted group of substances, which will alter the visual appearance of the second injectable fluid medication, and which have been approved for injection into human beings by the FDA.

Further, with respect to the above-noted distinguishment between the first injectable fluid medication and the second injectable fluid medication, by dissolving, respectively, the first selected substance and the second selected substance therein, the first injectable fluid medication and the second injectable fluid medication are generally the same volume, and the first and second selected substances are the same substance. The volume of the same substance dissolved in the first injectable fluid medication is less than the volume of the same substance dissolved in the second injectable fluid medication to produce different altered visual appearances between the first and second injectable fluid medications. In this manner, the first and second injectable fluid medicines are distinguishable by different shades of the same color.

In addition, with respect to the above-noted distinguishment between the first injectable fluid medication and the second injectable fluid medication, by dissolving, respectively, the first selected substance and the second selected substance therein, the initial visual appearance is a clear appearance, and the altered visual appearance thereof is a first non-clear appearance. The initial visual appearance of the second injectable fluid medication is a clear visual appearance, and the altered visual appearance thereof is a second non-clear appearance, which is different from the first non-clear visual appearance. The first altered visual appearance can be a first color, and the second altered visual appearance can be a second color different from the first color. Also, the first color and the second color can be the same, with a shade of the first color being different from a shade of the second color. Further, the form of each of the first selected substance and the second selected substance can be a fluid, a powder, or a solid.

This technique could be used where the first and second injectable fluid medications are different medications, but are in the different groups of the pharmaceuticals noted above. For example, in the above listing of the different groups of pharmaceuticals, barbiturates is one group with the medication phenobarbital, and opioids is another group with the medication morphine. Thus, even though the medications are in different groups of pharmaceuticals, different shades of the color can be used to identify the different medications in different groups, for example, a light shade of the color for phenobarbital, and a dark shade of the color for morphine.

This technique could be used where the first and second injectable fluid medications are different medications, but are in the same group of pharmaceuticals noted above. For example, in the above listing of the different groups of pharmaceuticals, phenobarbital and pentobarbital are different injectable fluid medications, but both are in the barbiturates group. Thus, a given color can be assigned in a color code scheme to identify a given group, for example, barbiturates, and different shades of the given color can be used to identify the different medications within the given group, for example, a light shade of the color for phenobarbital, and a dark shade of the color for pentobarbital.

In another technique, the volume of the first injectable fluid medication is less than the volume of the second injectable fluid medications by a prescribed ratio, and the first and second selected substances are the same substance. The volume of the same substance dissolved in the first injectable fluid medication is less than the volume of the same substance dissolved in the second injectable fluid medication by the prescribed ratio, to produce different altered visual appearances between the first and second injectable fluid medications.

The first injectable fluid medication and the first selected substance can be placed in a first two-compartment container in the manner noted above, and, in similar fashion, the second injectable fluid medication and the second selected substance can be placed in a second two-compartment container in the same manner as placement of the first medication and the first selected substance in the first two-compartment container.

In the context of the methods of preparing the coded injectable fluid medications, such methods are inherent in the description above regarding the identification of the coded injectable fluid medication, and regarding the distinguishment between two different coded injectable fluid medications. Also, the properties and various visual appearances of the medications and the properties of the substances, which are set forth herein in some of the method claims, are described above.

In the preparation of a coded injectable fluid medication, an injectable fluid medication, defined above, and a selected substance, also defined above, are provided. The selected substance is then dissolved in the medication. Further, two-compartment containers, each with releasable seals, are provided for segregated placement of the injectable fluid medications and the selected substances in the segregated compartments, with facilities attached to the containers to facilitate opening of the seals. Further, the seals are opened to facilitate dissolving the substances in the medications.

In the preparation of two coded injectable fluid medications for distinguishment therebetween, a first injectable fluid medication, a selected substance, and a second injectable medication are provided. Further, the selected substance is dissolved in the first injectable fluid medication. Also, a second selected substance is provided. The second selected substance is dissolved in the second injectable fluid medication.

As described above, and claimed below, a coded injectable fluid medication is identifiable by the visual appearance of the medication, and methods of preparing the coded medication are provided. Further, two, or more, coded injectable fluid medications are distinguishable by the difference in their visual appearances, and methods of preparing the two medications are provided. By use of the visual appearance of the coded injectable fluid medications, the effort to eliminate the medication-selection errors, which, in the past, have resulted in injury, and even death, to patients, is significantly enhanced.

In general, the above-identified embodiments are not to be construed as limiting the breadth of the present invention. Modifications, and other alternative constructions, will be apparent which are within the spirit and scope of the invention as defined in the appended claims.

1. A coded injectable fluid medication identifiable by a coded visual appearance thereof, for use in the treatment of patients, which comprises:
an injectable fluid medication having a prescribed efficacy and an initial visual appearance, selected from the classes of pharmaceuticals consisting of barbiturates, benzodiazepines, opioids, Etomidate and derivatives thereof, local anesthetics, muscle relaxants, cardiovascular medications, antihistamines, anticholinergics, anticholinesterases, antitoxins/antidotes, electrolytes, anticoagucants and its reversal agents, thrombolytics, butyrophenones, antibiotics, antiseizure medications, chemotherapeutics, contrast media, radiologic agents, tranquilizers, hormones, emetics, immunosuppressants, antineoplastics, cardiac glycosides, nonsteriodal anti-inflammatories, antiemetics, immunoregulants, vitamins, anticonvulsants, antiparasitics, diuretics, vaccines, antiarthritics, anti-inflammatories, sedatives, thiazines, hypnotics, respiratory stimulants, alpha-2 adrenergic antagonists, and antivenoms; and
a selected substance dissolved in the injectable fluid medication, without altering the prescribed efficacy of the injectable fluid medication, to produce an altered visual appearance of the injectable fluid medication which is different from the initial visual appearance, where the selected substance is selected from any one or more substances of a group of substances which will alter the visual appearance of the injectable fluid medication, and which have been approved by the Food and Drug Administration of the U.S. Department of Health and Human Services for injection into human beings.

2. The coded injectable fluid medication as set forth in claim 1, wherein the initial visual appearance of the injectable fluid medication is a clear appearance, and the altered visual appearance thereof is a non-clear appearance.

3. The coded injectable fluid medication as set forth in claim 2, wherein the altered visual appearance of the injectable fluid medication is a colored appearance.

4. The coded injectable fluid medication as set forth in claim 1, which further comprises:
the selected substance being selected from a group consisting of any one composition, or a mixture of two or more compositions, which, when dissolved in the injectable fluid medication will change the initial visual appearance of the injectable fluid medication to a selected color.

5. The coded injectable fluid medication as set forth in claim 1, wherein a form of the selected substance is selected from the group consisting of a fluid, a powder and a solid.

6. The coded injectable fluid medication as set forth in claim 1, which further comprises:
a container formed with a first compartment and a second compartment;
a releasible seal located in the container to preclude communication between the first compartment and the second compartment;
the injectable fluid medication being located in the first compartment of the container;
the selected substance being located in the second compartment of the container; and
means attached to the container for facilitating opening of the seal so that the selected substance dissolves in the injectable fluid medication to produce the altered visual appearance thereof.

7. A first coded injectable fluid medication and a second coded injectable fluid medication, which is a different medication from the first coded injectable fluid medication, coded by the visual appearances thereof, for distinguishing the first coded injectable fluid medication from the second coded injectable fluid medication, for use in the treatment of patients, which comprises:
a first injectable fluid medication having a prescribed efficacy and an initial visual appearance, and selected from the classes of pharmaceuticals consisting of barbiturates, benzodiazepines, opioids, anesthetics, Etomidate and derivatives thereof, local anesthetics, muscle relaxants, cardiovascular medications, antihistamines, anticholinergics, anticholinesterases, antitoxins/antidotes, electrolytes, anticoagucants and its reversal agents, thrombolytics, butyrophenones, antibiotics, antiseizure medications, chemotherapeutics, contrast media, radiologic agents, tranquilizers, hormones, emetics, immunosuppressants, antineoplastics, cardiac glycosides, nonsteriodal anti-inflammatories, antiemetics, immunoregulants, vitamins, anticonvulsants, antiparasitics, diuretics, vaccines, antiarthritics, steroidal anti-inflammatories, sedatives, thiazines, hypnotics, respiratory stimulants, alpha-2 adrenergic antagonists, and antivenoms;
a selected substance dissolved in the first injectable fluid medication, without altering the prescribed efficacy of the injectable fluid medication, to produce an altered visual appearance of the first injectable fluid medication which is different from the initial visual appearance of the first injectable fluid medication, where the selected substance is selected from any one or more substances of a group of substances which will alter the visual appearance of the injectable fluid medication, and which have been approved by the Food and Drug Administration of the U.S. Department of Health and Human Services for injection into human beings; and
a second injectable fluid medication having a prescribed efficacy and an initial visual appearance which is different from the altered visual appearance of the first injectable fluid medication, where the second injectable fluid medication is selected from the classes of pharmacuticals consisting of barbiturates, benzodiazepines, opioids, anesthetics, Etomidate and derivatives thereof, local anesthetics, muscle relaxants, cardiovascular medications, antihistamines, anticholinergics, anticholinesterases, antitoxins/antidotes, electrolytes, anticoagucants and reversal agents, thrombolytics, butyrophenones, antibiotics, antiseizure medications, chemotherapeutics, contrast media, radiologic agents, tranquilizers, hormones, emetics, immunosuppressants, antineoplastics, cardiac glycosides, nonsteriodal anti-inflammatories, antiemetics, immunoregulants, vitamins, anticonvulsants, antiparasitics, diuretics, vaccines, antiarthritics, anti-inflammatories, sedatives, thiazines, hypnotics, respiratory stimulants, alpha-2 adrenergic antagonists, and antivenoms.

8. The first coded injectable fluid medication and the second coded injectable fluid medication as set forth in claim 7, wherein the initial visual appearance of the first injectable fluid medication, and the initial visual appearance of the second injectable fluid medication are each a clear visual appearance.

9. The first coded injectable fluid medication and the second coded injectable fluid medication as set forth in claim 7, which further comprises:
the selected substance being selected from a group consisting of any one composition, or a mixture of two or more compositions, which, when dissolved in the first injectable fluid medication will change the initial visual appearance of the first injectable fluid medication to the altered visual appearance.

10. The first coded injectable fluid medication and the second coded injectable fluid medication as set forth in claim 7, which further comprises:
the selected substance being a first selected substance;
a second selected substance, different from the first selected substance, dissolved in the second injectable fluid medication, without altering the prescribed efficacy of the second injectable fluid medication, to produce an altered visual appearance of the second injectable fluid medication which is different from the initial visual appearance of the second injectable fluid medication, and different from the altered visual appearance of the first injectable fluid medication, where the second selected substance is selected from any one or more substances of a group of substances which will alter the visual appearance of the second injectable fluid medication, and which have been approved by the Federal Drug Administration of the U.S. Department of Health and Human Services for injection into human beings.

11. The first coded injectable fluid medication and the second coded injectable fluid medication as set forth in claim 10, which further comprises:
the second selected substance being selected from a group consisting of any one composition, or a mixture of two or more compositions, which, when dissolved in the second injectable fluid medication will change the initial visual appearance of the second injectable fluid medication to the altered visual appearance of the second injectable fluid medication.

12. The first coded injectable fluid medication and the second coded injectable fluid medication as set forth in claim 10, which further comprises:
the first injectable fluid medication and the second injectable fluid medication being of generally equal volume;
the first selected substance and the second selected substance being the same substance;
the volume of the same substance dissolved in the first injectable fluid medication being less than the volume of the same substance dissolved in the second injectable fluid medication to produce different altered visual appearances between the first injectable fluid medication and the second injectable fluid medication.

13. The first coded injectable fluid medication and the second coded injectable fluid medication as set forth in claim 10, which further comprises:
the first injectable fluid medication and the second injectable fluid medication being of generally equal volume and belonging to the same group of pharmaceuticals;
the first selected substance and the second selected substance being the same substance;
the volume of the same substance dissolved in the first injectable fluid medication being less than the volume of the same substance dissolved in the second injectable fluid medication to produce different altered visual appearances between the first injectable fluid medication and the second injectable medication.

14. The first coded injectable fluid medication and the second coded injectable fluid medication as set forth in claim 10, which further comprises:
the first injectable fluid medication being of a volume which is less than the volume of the second injectable fluid medication by a prescribed ratio;
the first selected substance and the second selected substance being the same substance;
the volume of the same substance dissolved in the first injectable fluid medication being less than the volume of the same substance dissolved in the second injectable fluid medication, by the prescribed ratio, to produce different altered visual appearances between the first injectable fluid medication and the second injectable fluid medication.

15. The first coded injectable fluid medication and the second coded injectable fluid medication as set forth in claim 10, wherein the initial visual appearance of the first injectable fluid medication is a clear appearance, and the altered visual appearance thereof is a first non-clear appearance, and the initial visual appearance of the second injectable fluid medication is a clear appearance and the altered visual appearance thereof is a second non-clear visual appearance different from the first altered non-clear appearance.

16. The first coded injectable fluid medication and the second coded injectable fluid medication as set forth in claim 10, wherein the altered visual appearance of the first injectable fluid medication is a first color, and the altered visual appearance of the second injectable fluid medication is a second color different from the first color.

17. The first coded injectable fluid medication and the second coded injectable fluid medication as set forth in claim 16, wherein the first color is the same as the second color, with a shade of the first color being different from a shade of the second color.

18. The first coded injectable fluid medication and the second coded injectable fluid medication as set forth in claim 10, wherein a form of the first selected substance and a form of the second selected substance are each selected from the group consisting of a fluid, a powder and a solid.

19. The first coded injectable fluid medication and the second coded injectable fluid medication as set forth in claim 10, which further comprises:
a first container formed with a first compartment and a second compartment;
a releasible seal located in the first container to preclude communication between the first compartment and the second compartment of the first container;
the first injectable fluid medication being located in the first compartment of the first container;
the first selected substance being located in the second compartment of the first container;
means attached to the first container for facilitating the opening of the seal so that the first selected substance dissolves in the first injectable fluid medication to produce the altered visual appearance thereof;
a second container formed with a first compartment and a second compartment;
a releasible seal located in the second container to preclude communication between the first compartment and the second compartment of the second container;
the second injectable fluid medication being located in the first compartment of the second container;
the second selected substance being located in the second compartment of the second container; and
means attached to the second container for facilitating the opening of the seal so that the second selected substance dissolves in the second injectable fluid medication to produce the altered visual appearance thereof.

20. A method for preparing a coded injectable fluid medication identifiable by the visual appearance thereof, for use in the treatment of patients, which comprises the steps of:
providing an injectable fluid medication having a prescribed efficacy and an initial visual appearance, selected from the classes of pharmaceuticals consisting of barbiturates, benzodiazepines, opioids, anesthetics, Etomidate and derivatives thereof, local anesthetics, muscle relaxants, cardiovascular medications, antihistamines, anticholinergics, anticholinesterases, antitoxins/antidotes, electrolytes, anticoagucants and its reversal agents, thrombolytics, butyrophenones, antibiotics, antiseizure medications, chemotherapeutics, contrast media, radiologic agents, tranquilizers, hormones, emetics, immunosuppressants, antineoplastics, cardiac glycosides, nonsteriodal anti-inflammatories, antiemetics, immunoregulants, vitamins, anticonvulsants, antiparasitics, diuretics, vaccines, antiarthritics, steriodal anti-inflammatories, sedatives, thiazines, hypnotics, respiratory stimulants, alpha-2 adrenergic antagonists, and antivenoms;
providing a selected substance which, when dissolved in the injectable fluid medication, without altering the prescribed efficacy of the injectable fluid medication, will produce an altered visual appearance of the injectable fluid medication which is different from the initial visual appearance, where the selected substance is selected from any one or more substances of a group of substances which will alter the visual appearance of the injectable fluid medication, and which have been approved by the Federal Drug Administration of the U.S. Department of Health and Human Services for injection into human beings; and
dissolving the selected substance in the injectable fluid medication to produce the altered visual appearance of the injectable fluid medication.

21. The method as set forth in claim 20, wherein the initial visual appearance of the injectable fluid medication is a clear visual appearance, and the altered visual appearance thereof is a non-clear visual appearance.

22. The method as set forth in claim 21, wherein the altered visual appearance of the injectable fluid medication is a color visual appearance.

23. The method as set forth in claim 20, wherein the selected substance is selected from a group consisting of any one composition, or a mixture of two or more compositions, which, when dissolved in the injectable fluid medication will change the initial visual appearance of the injectable fluid medication to a color visual appearance.

24. The method as set forth in claim 20 wherein a form of the selected substance is selected from the group consisting of a fluid, a powder and a solid.

25. The method as set forth in claim 20, which further comprises the steps of:
providing a container formed with a first compartment and a second compartment;
locating a releasible seal in the container to preclude communication between the first compartment and the second compartment;
placing the injectable fluid medication being in the first compartment of the container;
placing the selected substance in the second compartment of the container; and
attaching a facility to the container to facilitate selective opening of the seal.

26. The method as set forth in claim 25, which further comprises the step of:
opening the seal so that the selected substance mixes with, and dissolves in, the injectable fluid medication to produce the altered visual appearance thereof.

27. A method of preparing a first coded injectable fluid medication and a second coded injectable fluid medication, which is a different medication from the first coded injectable fluid medication, for distinguishing the first coded injectable fluid medication from the second coded injectable fluid medication, by the visual appearances thereof, for the treatment of patients, which comprises the steps of:
providing a first injectable fluid medication having a prescribed efficacy and an initial visual appearance, and selected from the classes of pharmaceuticals consisting of barbiturates, benzodiazepines, opioids, anesthetics, Etomidate and derivatives thereof, local anesthetics, muscle relaxants, cardiovascular medications, antihistamines, anticholinergics, anticholinesterases, antitoxins/antidotes, electrolytes, anticoagucants and its reversal agents, thrombolytics, butyrophenones, antibiotics, antiseizure medications, chemotherapeutics, contrast media, radiologic agents, tranquilizers, hormones, emetics, immunosuppressants, antineoplastics, cardiac glycosides, nonsteriodal anti-inflammatories, antiemetics, immunoregulants, vitamins, anticonvulsants, antiparasitics, diuretics, vaccines, antiarthritics, steroidal anti-inflammatories, sedatives, thiazines, hypnotics, respiratory stimulants, alpha-2 adrenergic antagonists, and antivenoms;
providing a selected substance which, when dissolved in the first injectable fluid medication, without altering the prescribed efficacy of the injectable fluid medication, will produce an altered visual appearance of the first injectable fluid medication which is different from the initial visual appearance of the first injectable fluid medication, where the selected substance is selected from any one or more substances of a group of substances which will alter the visual appearance of the injectable fluid medication, and which have been approved by the Federal Drug Administration of the U.S. Department of Health and Human Services for injection into human beings; and
providing a second injectable fluid medication having a prescribed efficacy and an initial visual appearance which is different from the altered visual appearance of the first injectable fluid medication, where the second injectable fluid medication is selected from the classes of pharmaceuticals consisting of barbiturates, benzodiazepines, opioids, anesthetics, Etomidate and derivatives thereof, local anesthetics, muscle relaxants, cardiovascular medications, antihistamines, anticholinergics, anticholinesterases, antitoxins/antidotes, electrolytes, anticoagucants and its reversal agents, thrombolytics, butyrophenones, antibiotics, antiseizure medications, chemotherapeutics, contrast media, radiologic agents, tranquilizers, hormones, emetics, immunosuppressants, antineoplastics, cardiac glycosides, nonsteriodal anti-inflammatories, antiemetics, immunoregulants, vitamins, anticonvulsants, antiparasitics, diuretics, vaccines, antiarthritics, steroidal anti-inflammatories, sedatives, thiazines, hypnotics, respiratory stimulants, alpha-2 adrenergic antagonists, and antivenoms.

28. The method as set forth in claim 27 which further comprises the step of:
dissolving the selected substance in the first injectable fluid medication to produce the altered visual appearance of the first injectable fluid medication, which is different from the initial visual appearance of the second injectable fluid medication.

29. The method as set forth in claim 27, wherein the initial visual appearance of the first injectable fluid medication, and the initial visual appearance of the second injectable fluid medication are each a clear visual appearance.

30. The method as set forth in claim 27, wherein the selected substance is selected from a group consisting of any one composition, or a mixture of two or more compositions, which, when dissolved in the first injectable fluid medication, will change the initial visual appearance of the first injectable fluid medication to the altered visual appearance.

31. The method as set forth in claim 27, wherein the selected substance is a first selected substance, which further comprises the step of:
providing a second selected substance, different from the first selected substance, which, when dissolved in the second injectable fluid medication, without altering the prescribed efficacy of the second injectable fluid medication, will produce an altered visual appearance of the second injectable fluid medication which is different from the initial visual appearance of the second injectable fluid medication, and different from the altered visual appearance of the first injectable fluid medication, where the second selected substance is selected from any one or more substances of a group of substances which will alter the visual appearance of the second injectable fluid medication, and which have been approved by the Federal Drug Administration of the U.S. Department of Health and Human Services for injection into human beings.

32. The method as set forth in claim 31 which further comprises the step of:
dissolving the second selected substance in the second injectable fluid medication to produce the altered visual appearance of the second injectable fluid medication, which is different from the altered visual appearance of the first injectable fluid medication.

33. The method as set forth in claim 31, wherein the second selected substance is selected from a group consisting of any one composition, or a mixture of two or more compositions, which, when dissolved in the second injectable fluid medication will change the initial visual appearance of the second injectable fluid medication to the altered visual appearance of the second injectable fluid medication.

34. The method as set forth in claim 31, wherein the first selected substance and the second selected substance are the same substance, which further comprises the steps of:
providing the first injectable fluid medication in a prescribed volume;
providing the second injectable fluid medication in a volume generally equal to the prescribed volume;
dissolving a first volume of the same substance in the first injectable fluid medication; being less than the volume of the same
dissolving a second volume of the same substance in the second injectable fluid medication;
wherein the first volume is less than the second volume to produce diffferent altered visual appearances between the first injectable fluid medication and the second injectable medication.

35. The method as set forth in claim 31, wherein the initial visual appearance of the first injectable fluid medication is a clear appearance, and the altered visual appearance thereof is a first non-clear appearance, and the initial visual appearance of the second injectable fluid medication is a clear appearance and the altered visual appearance thereof is a second non-clear visual appearance different from the first altered non-clear appearance.

36. The method as set forth in claim 31, wherein the altered visual appearance of the first injectable fluid medication is a first color, and the altered visual appearance of the second injectable fluid medication is a second color different from the first color.

37. The method as set forth in claim 36, wherein the first color is the same as the second color, with a shade of the first color being different from a shade of the second color.

38. The method as set forth in claim 31, wherein a form of the first selected substance and a form of the second selected substance are each selected from the group consisting of a fluid, a powder and a solid.

39. The method as set forth in claim 31, which further comprises the steps of:
providing a first container formed with a first compartment and a second compartment;
locating a releasible seal in the first container to preclude communication between the first compartment and the second compartment of the first container;
placing the first injectable fluid medication in the first compartment of the first container;
placing the first selected substance in the second compartment of the first container;
attaching a facility to the first container to facilitate selective opening of the seal;
providing a second container formed with a first compartment and a second compartment;
locating a releasible seal in the second container to preclude communication between the first compartment and the second compartment of the second container;
placing the second injectable fluid medication in the first compartment of the second container;
placing the second selected substance in the second compartment of the second container; and
attaching a facility to the second container to facilitate selective opening of the seal of the second container.

40. The method as set forth in claim 39, which further comprises the steps of:
opening the seal of the first container so that the first selected substance mixes with, and dissolves in, the first injectable fluid medication to produce the altered visual appearance thereof; and
opening the seal of the second container so that the second selected substance mixes with, and dissolves in, the second injectable fluid medication to produce the altered visual appearance thereof.
